(19) United States
(12) Patent Application Publication
(10) Pub. No.: US 2016/0317474 A1
(43) Pub. Date: Nov. 3, 2016

(54) Managing Visual Dysfunction or Loss of Vision for Diabetic Subjects

(71) Applicants: EMORY UNIVERSITY, Atlanta, GA (US); THE UNITED STATES GOVERNMENT REPRESENTED BY THE UNITED STATES DEPARTMENT OF VETERANS AFFAIRS, Washington, DC (US)

(72) Inventors: Moe Hein Aung, Atlanta, GA (US); Machelle T. Pardue, Atlanta, GA (US); P. Michael Iuvone, Decatur, GA (US)

(21) Appl. No.: 15/105,957

(22) PCT Filed: Dec. 17, 2014
(86) PCT No.: PCT/US2014/070707

§ 371 Date: Jun. 17, 2016

Related U.S. Application Data
(60) Provisional application No. 61/917,600, filed on Dec. 18, 2013.
(60) Provisional application No. 62/088,733, filed on Dec. 8, 2014.

(51) Int. Cl. A61K 31/137; A61K 45/06; A61K 9/00; A61K 31/198

(52) U.S. Cl. CPC A61K 31/137; A61K 31/198; A61K 45/06; A61K 9/0053; A61K 9/0048

(57) ABSTRACT

This disclosure relates to managing diabetes induced visual dysfunctions or vision loss by altering levels of dopamine. In certain embodiments, the disclosure relates to methods of treating or preventing visual dysfunction or loss of vision comprising administering an effective amount of dopamine, derivative, ester, prodrug, or salt thereof to a subject wherein the subject is at risk of, exhibiting symptoms of or diagnosed with diabetes or diabetic retinopathy.

### CROSS REFERENCE TO RELATED APPLICATIONS

This application claims priority to U.S. Provisional Application No. 61/917,600 filed Dec. 18, 2013 and U.S. Provisional Application No. 62/088,733 filed Dec. 8, 2014, both hereby incorporated by reference in their entirety.

### STATEMENT REGARDING FEDERALLY FUNDED RESEARCH

This invention was made with government support under P30 EY006360 and R01 EY004864 awarded by National Institutes of Health and E0951-R and C9257-5 awarded by Department of Veterans Affairs. The government has certain rights in the invention.

### BACKGROUND

Diabetic retinopathy (DR) is a leading cause of vision impairment in working-age adults. Laser surgery, injection of corticosteroids or VEGF antibodies into the eye, and vitrectomy are treatments for DR; however, they are not universally effective. Thus, there is a need to identify improved methods of managing DR.

Dopamine (DA) is a neurotransmitter in both the brain and retina. Historically, DR has been primarily considered a vascular disorder due to its association with late-stage structural defects of the retinal vasculature. Typically, after decades of hyperglycemia, increasing retinal vascular occlusions produce ischemia that drives an aggressive neovascularization response (also known as proliferative DR) and/or macular edema. These late-stage vascular lesions are the direct antecedents of severe vision loss associated with DR. Although clinical research has emphasized retinal vascular changes in diabetes, retinal neuronal dysfunction that predates clinically detectable vascular lesions is increasingly recognized. Electroretinogram (ERG) responses are consistently diminished and delayed in diabetic patients without vascular pathologies. Moreover, several neuronal cell types are less abundant in diabetic retinas compared with age-matched control retinas. In addition, diabetic patients with angiographically normal retinas experience subtle visual dysfunction, including abnormal color vision and decreased contrast sensitivity. Similar early visual dysfunctions have been consistently replicated in rodent models of diabetes. Although most reports emphasize the potential role of retinal defects as a contributing factor for the visual deficits, little is known about the underlying mediator(s).

Buttner et al. report L-DOPA improves color vision in Parkinson's disease. J Neural Transm Park Dis Dement Sect, 1994, 7(1):13-9. See also Leguire et al., Levodopa-carbidopa and childhood retinal disease, J AAPOS, 1998, 2(2):79-85 and US Published Application Number 2003/0069232.

Jackson et al. report retinal dopamine mediates multiple dimensions of light-adapted vision. See J Neurosci, 2012, 32:9359-9368.

Nishimura and Kuriyama report alterations in the retinal dopaminergic neuronal system in rats with streptozotocin-induced diabetes. J Neurochem, 1985, 45:448-455.

Gastinger et al. report loss of cholinergic and dopaminergic amacrine cells in streptozotocin-diabetic rat and Ins2Akita-diabetic mouse retinas. Invest Ophthalmol Vis Sci, 2006, 47:3143-3150.

Herrmann et al. report rod vision is controlled by dopamine-dependent sensitization of rod bipolar cells by GABA. Neuron, 2011, 72:101-110.

Aung et al., report a role of dopamine deficiency in visual dysfunction in early-stage diabetic retinopathy, ARVO 2013 Annual Meeting, May 5-9, 2013.

Aung et al. report dopamine deficiency contributes to early visual dysfunction in a rodent model of type 1 diabetes. J Neurosci. 2014, 34(3):726-36.

References cited herein are not an admission of prior art.

### SUMMARY

This disclosure relates to managing diabetes induced visual dysfunctions or vision loss by altering levels of dopamine. In certain embodiments, the disclosure relates to methods of treating or preventing visual dysfunction or loss of vision comprising administering an effective amount of dopamine, derivative, ester, prodrug, or salt thereof to a subject wherein the subject is at risk of, exhibiting symptoms of or diagnosed with diabetes or diabetic retinopathy.

In certain embodiments, the dopamine derivative is levodopa.

In certain embodiments, the dopamine, derivative, ester, prodrug, or salt thereof is administered in combination with an aromatic L-amino acid decarboxylase inhibitor. In certain embodiments, the aromatic L-amino acid decarboxylase inhibitor is selected from carbidopa, benserazide, methyldopa, and α-difluoromethyl-dopa

In certain embodiments, the dopamine, derivative, ester, prodrug, or salt thereof is administered in combination with a catechol-O-methyl transferase (COMT) inhibitor. In certain embodiments, catechol-O-methyl transferase (COMT) inhibitor is selected from entacapone, tolcapone, and nitecapone.

In certain embodiments, one administers an effective amount of levodopa in combination with carbidopa and entacapone.

In certain embodiments, levodopa is administered in combination with fluocinolone acetonide for the treatment of diabetic macular edema.

In certain embodiments, the dopamine, derivative, ester, prodrug, or salt thereof administered orally or into the vitreous or sclera of the eye, e.g., administered by an intravitreal injection or an implant. In certain embodiments, the compounds disclosed herein are in a liquid and are administered by the periocular (or transscleral) route that includes retrobulbar, peribulbar, subtenon and subconjunctival route by the use of microneedles or compound coated microneedles.

In certain embodiments, the dopamine, derivative, ester, prodrug, or salt thereof is administered in combination with another ocular agent selected from brimonidine, ganciclovir, anecortave, anecortave acetate, ranibizumab, bevacizumab, and squalamine or anti-inflammatory agent such as triamcinolone, triamcinolone acetonide, fluocinolone, fluocinolone acetonide, and dexamethasone.

In certain embodiments, the subject is a human.

In certain embodiments, the subject is administered dopamine, derivative, ester, prodrug, or salt thereof daily.

In certain embodiments, the disclosure relates to methods of treating or preventing visual dysfunction or loss of vision comprising administering an effective amount of a dopamine receptor agonist to a subject wherein the subject is at risk of, exhibiting symptoms of, or diagnosed with diabetes or diabetic retinopathy.

In certain embodiments, the dopamine receptor agonist is ropinirole or pramipexole, derivative, ester, prodrug, or salt thereof.

In certain embodiments, the dopamine, derivative, ester, prodrug, or salt thereof administered orally or into the vitreous or sclera of the eye, e.g., administered by an intravitreal injection or an implant. In certain embodiments, the compounds disclosed herein are in a liquid and are administered into the suprachoroidal space by the use of microneedles or compound coated microneedles.

In certain embodiments, the dopamine receptor agonist is administered in combination with dopamine, derivative, ester, prodrug, or salt thereof optionally in combination with an aromatic L-amino acid decarboxylase inhibitor and/or a catechol-O-methyl transferase (COMT) inhibitor.

In certain embodiments, one administers an effective amount of ropinirole or pramipexole in combination with levodopa optionally in combination with carbidopa and entacapone.

In certain embodiments, the dopamine receptor agonist is administered in combination with another ocular agent selected from anecortave, anecortave acetate, ranibizumab, bevacizumab, and squalamine.

In certain embodiments, the subject is a human.

In certain embodiments, the subject is administered the dopamine receptor agonist daily.

In certain embodiments, the dopamine derivative is levodopa.

In certain embodiments, dopamine receptor agonist is a D1 or D4 receptor agonist. In certain embodiments, dopamine receptor agonist is ropinirole, derivative, ester, prodrug, or salt thereof. In certain embodiments, the dopamine receptor agonist is pramipexole, derivative, ester, prodrug, or salt thereof.

In certain embodiments, the disclosure relates to methods of treating or preventing visual dysfunction or loss of vision comprising administering an effective amount of a dopamine receptor agonist to a subject wherein the subject is at risk of, exhibiting symptoms of or diagnosed with diabetic retinopathy, Type I diabetes, Type II diabetes or combinations thereof.

### BRIEF DESCRIPTION OF THE FIGURES

FIG. 1A shows data indicating diabetes results in reduced retinal DA content. Overall, DM rats exhibited significantly reduced DA levels compared with CTRL animals (main treatment effect: p<0.001). There was also a significant age-dependent increase in DA levels of all animals (main duration effect: p<0.01). Control is on the left and DM is on the right.

FIG. 1B shows data indicating regardless of diabetes status, rats had significantly higher DOPAC levels at the 12-week time point than at the 4-week time point (main duration effect: p<0.001). DM animals showed a trend toward lower DOPAC levels compared with CTRL animals at the 12-week time point. Control is on the left and DM is on the right.

FIG. 1C shows data indicating metabolism of DA to DOPAC did not differ between CTRL and DM animals. No significant change in the DOPAC/DA ratio was detected due to diabetes in Long-Evans rats. However, there was an increase in dopamine metabolism due to age (main duration effect: p=0.001). Control is on the left and DM is on the right.

FIG. 2A shows data indicating diabetes lowers retinal DA levels in STZ-induced DM mice. At the 5-week time point, DM mice had significantly reduced retinal DA contents compared with the CTRL mice. Control is on the left and DM is on the right.

FIG. 2B shows data indication a slight trend for decreased DOPAC levels. Control is on the left and DM is on the right.

FIG. 2C shows data indication a slight trend for decreased DOPAC/DA ratios. Control is on the left and DM is on the right.

FIG. 3A shows data indicating chronic L-DOPA treatment delays early diabetes-induced visual dysfunction. DMWT and Veh mice showed significant reductions in spatial frequency threshold from CTRL animals as early as 3 weeks after STZ. In contrast, the visual deficits appeared in DM WT and L-DOPA mice starting at 4 weeks after STZ with a slower and less severe progression.

FIG. 3B shows data indicating contrast sensitivities were significantly reduced in DMWT and Veh mice at 4 weeks after STZ, whereas DMWT and L-DOPA mice only exhibited a slight decrease in sensitivity at 6 weeks after STZ. The asterisk indicates the treatment group for which significance was reached, with the exception of the asterisk, which refers to both CTRL WT and Veh and CTRL WT and L-DOPA groups.

FIG. 4A shows data indicating that a genetic model of retinal DA deficiency (rTHKO) replicates early diabetes-induced visual dysfunction and could be rescued with L-DOPA treatment. Diabetes did not further impair the spatial frequency threshold levels of DM rTHKO and Veh mice and the spatial frequency thresholds of the DM WT and Veh and DM rTHKO and Veh groups became indistinguishable starting at 3 weeks after STZ.

FIG. 4B shows data indicating that the combination of rTHKO and diabetes did not further reduce contrast sensitivity within the time frame of this study and contrast sensitivities of DM WT and Veh and DM rTHKO and Veh mice were indistinguishable from 3 weeks after STZ onward.

FIG. 4C shows data indicating chronic L-DOPA treatment restored the spatial frequency thresholds of rTHKO mice (DM rTHKO+L-DOPA). DM rTHKO+L-DOPA mice had significantly higher spatial frequency thresholds than DM rTHKO+Veh mice throughout the study duration (post hoc comparison, p<0.001). Moreover, L-DOPA treatment in rTHKO mice significantly delayed the onset and slowed the progression of diabetes-induced impairment of spatial frequency threshold, which was only significant at 6 weeks after STZ.

FIG. 4D shows data indicating DM rTHKO+L-DOPA mice exhibited significantly better contrast sensitivities than DM rTHKO+Veh. The severity of perturbations in contrast sensitivity due to diabetes was also diminished in DM rTHKO+L-DOPA mice compared with DM WT+Veh mice. The asterisk indicates the treatment group for which significance was reached.

FIG. 5A shows data indicating changes in DA levels due to diabetes affect light-adapted retinal function. Representative raw waveforms to flicker stimuli (6 Hz) from (relative from top to bottom) CTRL WT, DM WT+Veh, DM WT+L-DOPA, DM rTHKO+Veh, and DM rTHKO+L-DOPA at the 5-week time point. The gray line indicates the peak of the response in a CTRL WT mouse; gray arrows indicate the peak of the response when delayed.

FIG. 5B shows data on average amplitudes of the flicker responses from experimental groups at the 5-week time point for CTRL WT, DM WT+Veh, DM WT+L-DOPA, DM rTHKO+Veh, and DM rTHKO+L-DOPA (relative from left to right).

FIG. 5C shows data on average implicit times of the flicker responses from experimental groups at the 5-week time point for CTRL WT, DM WT+Veh, DM WT+L-DOPA, DM rTHKO+Veh, and DM rTHKO+L-DOPA (relative from right to left). DM WT+Veh mice had reduced and delayed ERG responses compared with CTRL WT mice. L-DOPA treatment was able to restore ERG responses of DM WT mice to those of CTRL mice. Moreover, DM rTHKO+Veh mice with presumed lower DA content had severely reduced amplitudes than all other groups except that of DM WT+Veh animals. DM rTHKO+Veh mice also exhibited delayed responses from CTRL WT and DM WT+L-DOPA animals. The asterisk indicates the treatment group in which significance was reached.

FIG. 6A shows data indicating changes in DA levels due to diabetes affect dark-adapted retinal function. Representative raw waveforms in response to a dim-flash stimulus (−1.8 log cd s/m2) at the 5-week time point.

FIG. 6B shows average b-wave implicit times in response to a dim-flash stimulus (−1.8 log cd s/m2) at the 5-week time point for CTRL WT, DM WT+Veh, DM WT+L-DOPA, DM rTHKO+Veh, and DM rTHKO+L-DOPA (relative from left to right).

FIG. 6C shows representative raw waveforms in response to a bright-flash stimulus (0.6 log cd s/m2) at the 5-week time point. The gray lines indicate the peak of the b-wave in a CTRL WT mouse; gray arrows indicate the peak of the response when delayed. DM WT+Veh mice exhibited significantly delayed b-wave responses elicited with both dim and bright flash stimuli compared with CTRL WT and DM WT+L-DOPA mice.

FIG. 6D shows average b-wave implicit times in response to a bright-flash stimulus (0.6 log cd s/m2) at the 5-week time point for CTRL WT, DM WT+Veh, DM WT+L-DOPA, DM rTHKO+Veh, and DM rTHKO+L-DOPA (relative from left to right). L-DOPA treatment was able to restore ERG responses of DM WT mice (DM WT+L-DOPA) to those of CTRL WT mice. Similarly, DM rTHKO+Veh mice had severely delayed responses from CTRL WT and DM WT+L-DOPA animals at the bright flash stimulus. The asterisk indicates the treatment group in which significance was reached.

FIG. 7 shows data on mRNA levels of the examined dopaminergic system-related genes for CTRL WT, DM WT+Veh, DM WT+L-DOPA, DM rTHKO+Veh, and DM rTHKO+L-DOPA (relative from left to right). rTHKO mice had significantly lower expressions of Th than CTRL WT mice (t test, p<0.01). Diabetes in DM WT mice did not induce a significant change in mRNA levels of Th, Drd1, or Drd4 compared with the CTRL WT mice. Interestingly, L-DOPA treatment caused a downregulation of Drd4 (t test, p<0.05), but not of Drd1, in DM WT mice compared with CTRL WT mice. Note that the y-axis refers to averaged 2−ΔCt values, with ΔCt calculated by subtracting cycle threshold (Ct) of 18S from Ct of gene of interest (GOI). The asterisk indicates significant difference between the respective treatment group and the CTRL WT group.

FIG. 8A shows data indicating an improvement in OKT responses of 8-week DM WT mice after treatments with selective dopamine receptor agonists. Spatial frequency thresholds of DM WT mice improved significantly when treated with D1 receptor agonist. Treatment with the D4 receptor agonist failed to improve spatial frequency thresholds. DM+Veh, DM+D1R agonist, DM+D4R agonist (respectively from left to right).

FIG. 8B shows data indicating DM WT mice showed significantly enhanced contrast sensitivity levels only when treated with D4 agonist. However, neither treatment restored visual functions (spatial frequency threshold and contrast sensitivity) of DM WT mice to CTRL WT levels, indicated by the dashed lines with their variance (±SEM) represented by gray boxes. DM+Veh, DM+D1R agonist, DM+D4R agonist (respectively from left to right).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In certain embodiments, a pharmaceutical agent, which may be in the form of a salt or prodrug, is administered in methods disclosed herein that is specified by a weight. This refers to the weight of the recited compound. If in the form of a salt or prodrug, then the weight is the molar equivalent of the corresponding salt or prodrug.

It must be noted that, as used in the specification and the appended claims, the singular forms “a,” “an,” and “the” include plural referents unless the context clearly dictates otherwise.

“Subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulphur atom or replacing an amino group with a hydroxyl group. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, — NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

### Dopamine Deficiency Contributes to Early Visual Dysfunction

Data herein indicates that reduced retinal DA content is an underlying factor for the early visual deficits observed in DR. These data support a hypothesis that dysfunction in the neural retina due to diabetes is a contributing factor to visual dysfunction because L-DOPA treatment significantly improved retinal function and thereby vision. It was discovered that restoring DA levels (L-DOPA) or activating DA pathways (DA receptor agonists) in the retina serves as therapeutic interventions for DR.

Assessments in diabetic rodents and postmortem analyses of the dopaminergic pathways in retinal tissues were performed to examine the overall role of retinal DA in early visual deficits due to diabetes. Data indicates that diabetes produced retinal DA deficiency and that pharmacologic replacement of DA or stimulation of DA receptors ameliorated diabetes-associated visual dysfunction.

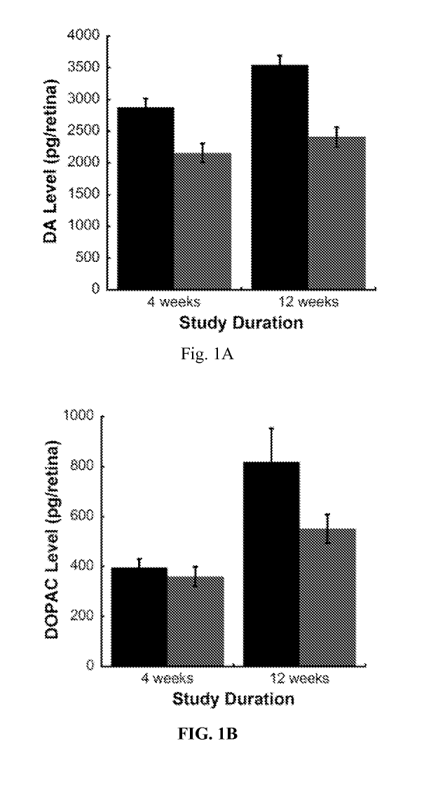

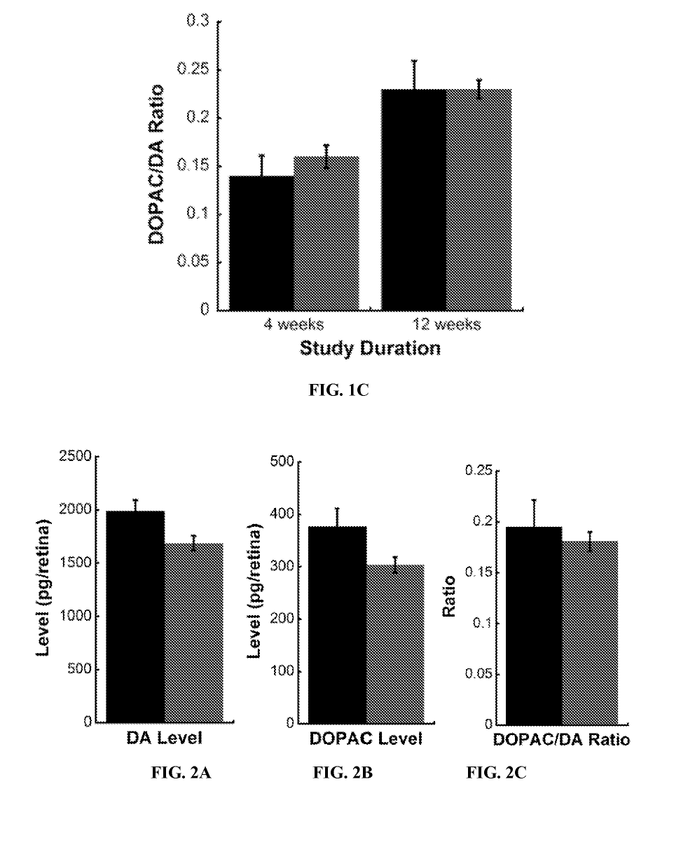

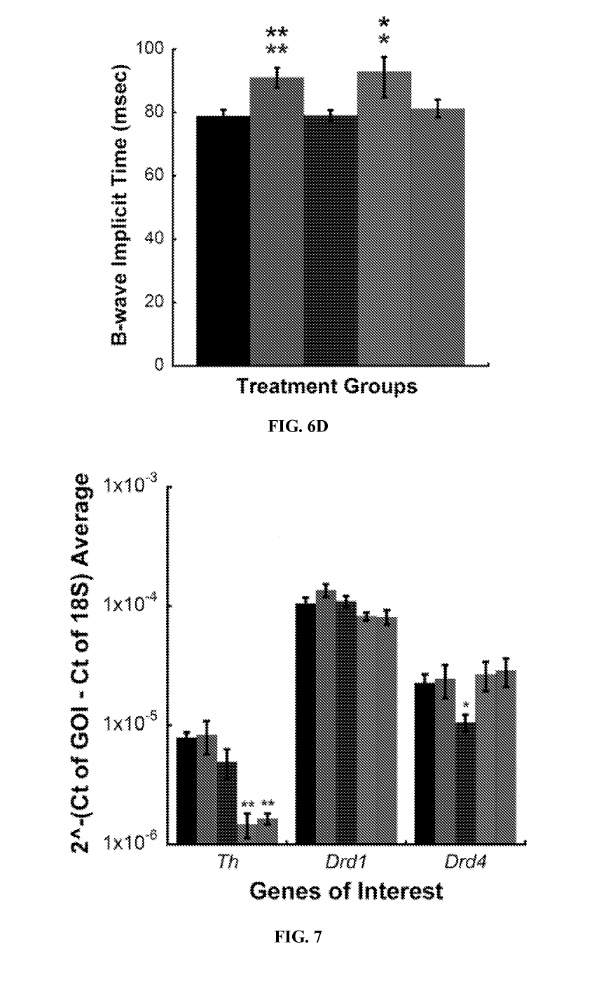

Studies indicate that reduced retinal DA contents in both DM rats and mice as early as 4-5 weeks after STZ (FIGS. 1, 2). Dysregulation at multiple sites of DA metabolic processing has been postulated to reduce DA abundance, but published results show inconsistencies. Although it is not intended that embodiments of this disclosure be limited by any particular mechanism, it is possible that retinal DA reduction in diabetes originates from decreased biosynthesis. Several studies have documented diminished L-DOPA accumulations in diabetic retinas with normal activity levels of TH, thereby concluding that DA deficiency is due to reduced tyrosine level in the retina. However, others indicate that DA alteration is due to decreased TH activity, leading to reduced rate of tyrosine hydroxylation. Although the Th transcript levels did not change due to diabetes in the experiments (FIG. 7), some studies have suggested that retinal TH protein levels are downregulated, presumably from increased posttranslational processing of the TH proteins or apoptotic loss of dopaminergic neurons. These inconsistencies may be attributed to animal strain differences, variable glycemic controls, or different durations of hyperglycemia across studies. A second plausible mechanism for reduced DA levels in diabetic animals is enhanced turnover of DA. Although DA turnover rates were not measures directly, metabolism of DA to DOPAC (DOPAC/DA ratio), an indirect assessment of DA turnover, was not altered by diabetes (FIGS. 1, 2). Mitochondrial dysfunction and oxidative stress associated with diabetes may also result in enhanced oxidation of DA and render it inactive biologically.

Aside from dysfunction of the dopaminergic neurons, disturbances in light transmission and/or retinal circadian clock pathways may alter retinal DA content because retinal DA synthesis and release are tightly regulated by such. Because cataract is a common complication of diabetes increased opacity of the lens could attenuate light transmission to the retina and thereby impair light-induced upregulation of DA in diabetic retinas. However, retinal DA levels were reduced starting at 4 weeks after STZ (FIG. 1A), before significant development of cataract, at 6 weeks in STZ-induced diabetic rats. Moreover, despite the lack of cataract development in our STZ mice at the 5-week time point, reduced retinal DA levels were found in these mice (FIG. 2). Therefore, diminished light input is not a likely contributing factor for early diabetes-induced retinal DA deficiency. In regard to altered circadian clocks underling DA deficiency in diabetes, studies have shown that both retinal and peripheral circadian clocks are dysfunctional in diabetic animals and mice with circadian clock mutations recapitulate diabetic phenotypes.

Experiments herein indicate that disruptions in the retinal dopaminergic system due to diabetes produce visual deficits. Not only does the onset of DA reductions coincide with the onset of visual defects, the results from L-DOPA and DA receptor agonist treatments provide evidence for the causal role of DA deficiency in visual loss in early-stage DR. These results are consistent with studies on multiple disorders with known abnormalities in dopamine signaling, such as Parkinson's disease and retinitis pigmentosa.

Data on the retinal-specific ERG results and the use of rTHKO mice corroborate the role of diminished retinal DA bioavailability in visual dysfunction. The effect is unlikely due to degeneration of retinal neurons because the examined time points were before reports of retinal degeneration in diabetes. Furthermore, no evidence of retinal degeneration was observed due to deletion of Th in the original characterization of the rTHKO mice. However, the loss of retinal DA in these mice was incomplete. The retinal DA levels and the numbers of TH-positive cells in the rTHKO mice were ˜10% of wild-type controls, similar to the DA levels observed in the experiments herein. Therefore, there may be a small number of dopaminergic amacrine cells in which the gene was not disrupted, resulting in a low level of Th expression (FIG. 7). It is unknown whether complete elimination of retinal DA would have a more severe effect on cell survival, retinal function, and ultimately visual function.

### Methods of Use

This disclosure relates to managing diabetes mellitus induced visual dysfunctions or vision loss by altering levels of dopamine. As used herein the term “diabetes” refers to diabetes mellitus which can be type 1 or type 2 diabetes, or gestational diabetes. Type 1 refers to a subject that fails to produce sufficient insulin. Type 2 refers to subjects that become resistant to insulin. Diabetes mellitus results in persistent hyperglycemia that produces reversible and irreversible pathologic changes within the microvasculature of various organs. Diabetics often develop visual dysfunctions such as diabetic retinopathy, glaucoma, cataracts, macular edema, abnormal color vision, and decreased contrast sensitivity. Diabetic retinopathy is traditionally characterized as a retinal microvascular disease that is manifested as a cascade of stages with increasing levels of severity and worsening prognoses for vision. Major risk factors reported for developing diabetic retinopathy include the duration of diabetes mellitus, quality of glycemic control, and presence of systemic hypertension.

Pathologic ocular neovascularization (NV) and related conditions are believed to occur as a cascade of events that progresses from an initiating stimulus to the formation of abnormal new capillaries. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

Retinal NV is observed in retinal ischemia, proliferative and nonproliferative diabetic retinopathy (PDR and NPDR, respectively), retinopathy of prematurity (ROP), central and branch retinal vein occlusion, and wet age-related macular degeneration (AMD). The retina includes choriocapillaries that form the choroid and are responsible for providing nourishment to the retina, Bruch's membrane that acts as a filter between the retinal pigment epithelium (RPE) and the choriocapillaries, and the RPE that secretes angiogenic and anti-angiogenic factors responsible for, among many other things, the growth and recession of blood vessels.

Neovascularization also occurs in a type of glaucoma called neovascular glaucoma in which increased intraocular pressure is caused by growth of connective tissue and new blood vessels upon the trabecular meshwork. Neovascular glaucoma is a form of secondary glaucoma caused by neovascularization in the chamber angle.

This disclosure relates to managing diabetes induced visual dysfunctions or vision loss by altering levels of dopamine. Increasing dopamine levels may improve ocular neovascularization for subjects with diabetes. Thus, in certain embodiments, the disclosure relates to treating or preventing retinal ischemia, proliferative and nonproliferative diabetic retinopathy (PDR and NPDR, respectively), retinopathy of prematurity (ROP), central and branch retinal vein occlusion, glaucoma, cataract, and age-related macular degeneration (AMD) by administering an effective amount of a dopamine receptor agonist or dopamine, derivative, ester, prodrug, or salt thereof e.g., levodopa optionally in combination with other agents reported herein to a subject in need thereof.

In certain embodiments, a subject may be in need thereof because the subject has recurrent abnormal blood sugar levels, diabetes, prediabetes, or recurrent abnormally high blood sugar levels. A normal fasting (no food for eight hours) blood sugar level is between 70 and 99 mg/dL. A normal blood sugar level two hours after eating is less than 140 mg/dL. Recurrent abnormal levels may be for more than a month, or more than three months, or more than six months, or more than a year.

In certain embodiments, the subject is diagnosed with diabetes or pre-diabetes. Diabetes is typically diagnosed by an indication of abnormally high blood sugar levels. Some examples include: two consecutive fasting blood glucose tests that are equal to or greater than 126 mg/dL; any random blood glucose that is greater than 200 mg/dL; A1c test, i.e., measure of a percentage of the glycated hemoglobin, that is equal to or greater than 6.5 percent; or a two-hour oral glucose tolerance test with any value over 200 mg/dL. Pre-diabetes is typically diagnosed by a higher than normal blood sugar level below the amounts indicated above. Some examples include: a fasting blood glucose in between 100-125 mg/dL; an A1c between 5.7-6.4 percent; and between 140 mg/dL and 199 mg/dL during a two-hour 75 g oral glucose tolerance test.

In certain embodiments, the disclosure relates to treating or preventing retinal ischemia, proliferative and nonproliferative diabetic retinopathy (PDR and NPDR, respectively), retinopathy of prematurity (ROP), central and branch retinal vein occlusion, and age-related macular degeneration (AMD) by administering an effective amount of a dopamine receptor agonist or dopamine, derivative, ester, prodrug, or salt thereof e.g., levodopa optionally in combination with other agents reported herein to a subject in need thereof, further in combination with other ocular agents including fluocinolone, fluocinolone acetonide, anecortave, anecortave acetate, ranibizumab bevacizumab, or squalamine.

In certain embodiments, the disclosure contemplates administering levodopa, and optional combinations disclosed herein, in combination with fluocinolone acetonide for uses reported herein or for the treatment of diabetic macular edema or posterior uveitis.

In certain embodiments, the disclosure contemplates administering levodopa, and optional combinations disclosed herein, in combination with dexamethasone for uses reported herein or for the treatment of macular edema.

In certain embodiments, the disclosure contemplates administering levodopa, and optional combinations disclosed herein, in combination with ganciclovir for uses reported herein or for the treatment of cytomegalo virus retinitis.

In certain embodiments, the dopamine derivative is levodopa, i.e., (−)-L-α-amino-β-(3,4-dihydroxybenzene)propanoic acid or salts thereof, that are administered by 100 to 250 mg or 250 to 500 mg, two or more or four or more times a day; the daily dosage may be increased by an additional 100 to 750 mg.

In certain embodiments, the dopamine, derivative, ester, prodrug, or salt thereof is administered in combination with an aromatic L-amino acid decarboxylase inhibitor. In certain embodiments, the aromatic L-amino acid decarboxylase inhibitor is selected from carbidopa, i.e., (−)-L-α hydrazino-α-methyl-β-(3,4-dihydroxybenzene), benserazide, methyldopa, and α-Difluoromethyl-DOPA.

In certain embodiments, the dopamine, derivative, ester, prodrug, or salt thereof is administered in combination with a catechol-O-methyl transferase (COMT) inhibitor. In certain embodiments, the catechol-O-methyl transferase (COMT) inhibitor is selected from entacapone, i.e., (E)-2-cyano-3-(3,4-dihydroxy-5-nitrophenyl)-N,N-diethyl-2-propenamide, tolcapone, and nitecapone.

In certain embodiments, the disclosure relates to a method of treating or preventing diabetic retinopathy comprising administering an effective amount of levodopa optionally in combination with carbidopa and entacapone. In certain embodiments, the daily or bi-daily administration includes a product that contains 10 to 15 mg of carbidopa, 25 to 75 mg of levodopa and 100 to 300 mg of entacapone. In certain embodiments, the daily or bi-daily administration includes a product that contains 15 to 30 mg of carbidopa, 50 to 100 mg of levodopa and 100 to 300 mg of entacapone. In certain embodiments, the daily or bi-daily administration includes a product that contains 20 to 40 mg of carbidopa, 75 to 125 mg of levodopa and 100 to 300 mg of entacapone. In certain embodiments, the daily or bi-daily administration includes a product that contains 20 to 40 mg of carbidopa, 125 to 175 mg of levodopa and 100 to 300 mg of entacapone. In certain embodiments, the daily or bi-daily administration includes a product that contains 25 to 75 mg of carbidopa, 100 to 300 mg of levodopa, and 100 to 300 mg of entacapone.

In certain embodiments, the disclosure relates to a method of treating or preventing diabetic retinopathy comprising administering an effective amount of levodopa in combination with carbidopa. In certain embodiments, the daily or bi-daily administration includes a product that contains 10 to 15 mg of carbidopa and 25 to 75 mg of levodopa. In certain embodiments, the daily or bi-daily administration includes a product that contains 15 to 30 mg of carbidopa and 50 to 100 mg of levodopa. In certain embodiments, the daily or bi-daily administration includes a product that contains 20 to 40 mg of carbidopa and 75 to 125 mg of levodopa. In certain embodiments, the daily or bi-daily administration includes a product that contains 20 to 40 mg of carbidopa and 125 to 175 mg of levodopa. In certain embodiments, the daily or bi-daily administration includes a product that contains 25 to 75 mg of carbidopa and 100 to 300 mg of levodopa.

In certain embodiments, the dopamine, derivative, ester, prodrug, or salt thereof is administered in combination with a dopamine receptor agonist. In certain embodiments, the dopamine receptor agonist is a D1, D2, D3, or D4 receptor agonist.

In certain embodiments the D1 receptor agonist is 1-Phenyl-2,3,4,5-tetrahydro-(1H)-3-benzazepine-7,8-diol, derivatives or salts thereof. Other contemplated D1 receptor agonists are 2,3,4,5-Tetrahydro-1-phenyl-3-(2-propenyl)-1H-3-benzazepine-7,8-diol, 1-(aminomethyl)-3,4-dihydro-3-phenyl-1H-2-benzopyran-5,6-diol, 1-(Aminomethyl)-3,4-dihydro-3-tricyclo[3.3.1.13,7]dec-1-yl-[1H]-2-benzopyran-5,6-diol, 4,6,6a,7,8,12b-Hexahydro-7-methylindolo[4,3-a]phenanthridine, 10,11-Dihydroxy-5,6,6a,7,8,12b-hexahydrobenzo[a]phenanthridine, 6-Chloro-2,3,4,5-tetrahydro-1-(4-hydroxyphenyl)-1H-3-benzazepine-7,8-diol, (N)-1-(2-Nitrophenyl)ethylcarboxy-3,4-dihydroxyphenethylamine, 6-Chloro-2,3,4,5-tetrahydro-1-phenyl-1H-3-benzazepine, 6-Chloro-2,3,4,5-tetrahydro-1-(3-methylphenyl)-3-(2-propenyl)-1H-3-benzazepine-7,8-diol, and 6-Chloro-2,3,4,5-tetrahydro-3-methyl-1-(3-methylphenyl)-1H-3-benzazepine-7,8-diol, derivatives or salts thereof.

In certain embodiments the D4 receptor agonist is N-(Methyl-4-(2-cyanophenyl)piperazinyl-3-methylbenzamide, derivatives or salts thereof. Other contemplates D4 agonist include N-(3-Methylphenyl)-4-(2-pyridinyl)-1-piperidineacetamide, 2-[[4-(2-Pyridinyl)-1-piperazinyl]methyl]-1H-benzimidazole, 5-[(3,6-Dihydro-4-phenyl-1(2H)-pyridinyl)methyl]-2-methyl-4-pyrimidinamine, and N-[2-[4-(2-Methoxyphenyl)-1-piperazinyl]ethyl]-N-2-pyridinylcyclohexanecarboxamide, derivatives or salts thereof.

In certain embodiments, method of treating or preventing visual dysfunction or loss of vision comprising administering an effective amount of a dopamine receptor agonist to a subject wherein the subject is at risk of, exhibiting symptoms of or diagnosed with diabetic retinopathy, glaucoma, cataract, macular edema, Type I diabetes, Type II diabetes or combinations thereof. In certain embodiments, the subject is a human.

In certain embodiments, the dopamine receptor agonist is fenoldopam, bromocriptine, cabergoline, ciladopa, dihydrexidine, dinapsoline, doxanthrine, epicriptine, lisuride, pergolide, piribedil, pramipexole, propylnorapomorphine, quinagolide, ropinirole, rotigotine, roxindole, sumanirole or combinations thereof.

In certain embodiments, the disclosure contemplates administering dopamine receptor agonist, and optional combinations disclosed herein, in combination with fluocinolone acetonide for uses reported herein or for the treatment of diabetic macular edema or posterior uveitis.

In certain embodiments, the disclosure contemplates administering dopamine receptor agonist, and optional combinations disclosed herein, in combination with dexamethasone for uses reported herein or for the treatment of macular edema.

In certain embodiments, the disclosure contemplates administering dopamine receptor agonist, and optional combinations disclosed herein, in combination with ganciclovir for uses reported herein or for the treatment of cytomegalo virus retinitis.

In certain embodiments, the dopamine, derivative, ester, prodrug, or salt thereof administered orally or into the vitreous or sclera of the eye, e.g., administered by an intravitreal injection or an implant. In certain embodiments, the dopamine receptor agonist or dopamine, derivative, ester, prodrug, or salt thereof is administered orally or by an intravitreal injection or an implant, e.g., surgical administration of drug-loaded solid implants within the scleral tissue (i.e. intrascleral delivery).

In certain embodiments, the compositions comprising dopamine, derivative, ester, prodrug, or salt thereof or dopamine receptor agonist as reported herein is administered in a liquid or gel composition into the vitreous cavity of the eye.

In certain embodiments, the compounds disclosed herein are in a liquid and are administered by the periocular (or transscleral) route that includes retrobulbar, peribulbar, subtenon and subconjunctival route through the use of microneedles or compound coated microneedles.

The suprachoroidal space is a space between the sclera and choroid that goes circumferentially around the eye. In certain embodiments, the compounds disclosed herein are in a liquid and are administered into the suprachoroidal space by the use of microneedles or compound coated microneedles. Microneedles typically have an inner diameter of about 0.5 to 1.0 mm and an outer diameter of 1.0 to 2.0 mm.

In certain embodiments, the compounds are administered by periocular deposits on the outer surface of the globe.

### Formulations

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Depending upon the manner of introduction, the compounds described herein may be formulated in a variety of ways. Formulations containing one or more compounds can be prepared in various pharmaceutical forms, such as granules, tablets, capsules, suppositories, powders, controlled release formulations, suspensions, emulsions, creams, gels, ointments, salves, lotions, or aerosols and the like. Preferably, these formulations are employed in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. In another embodiment, the formulation is administered topically. Suitable topical formulations include, but are not limited to, lotions, ointments, creams, and gels. In a preferred embodiment, the topical formulation is a gel. In another embodiment, the formulation is administered intranasally.

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicon dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The concentration of the compound(s) to carrier and/or other substances may vary from about 0.5 to about 100 wt. % (weight percent). For oral use, the pharmaceutical formulation will generally contain from about 5 to about 100% by weight of the active material. For other uses, the pharmaceutical formulation will generally have from about 0.5 to about 50 wt. % of the active material.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multilayer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The formulation can provide pulsatile delivery of the one or more compounds. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In one embodiment, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule may comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

The compound described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastrointestinal drugs, muscle relaxants, nutritional agents, vitamins, parasympathomimetics, stimulants, anorectics and anti-narcoleptics. “Adjunctive administration”, as used herein, means the compound can be administered in the same dosage form or in separate dosage forms with one or more other active agents.

Specific examples of compounds that can be adjunctively administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

The additional active agent(s) can be formulated for immediate release, controlled release, or combinations thereof.

In certain embodiments, for ocular implants the compounds are in a matrix of polylactic acid (PLA), polyglycolic acid (PGA), poly(lactic-co-glycolic acid) (PLGA), polycaprolactones (PCL), polyanhydrides (PA) and polyortho esters (POE), poloxamer or combination thereof, configured for administered through hollow microneedle, e.g., 20 to 35 G, such as 22, 25, 27, 29 and 30 G microneedles, providing sustained release implants in the vitreous, sclera tissue, or sub-conjuctiva. Poloxamers are triblock copolymers composed of a central hydrophobic chain of polyoxypropylene (poly(propylene oxide)) flanked by two hydrophilic chains of polyoxyethylene (poly(ethylene oxide)) of varying molecular weights.

Other contemplated ocular implants include polyvinyl alcohol (PVA), ethylene vinyl acetate (EVA), silicon, and combinations thereof e.g., a combination of PVA with either EVA or silicon. In certain embodiments the polymer comprises polymethyl methacrylate (PMMA), tri(ethyleneglycol)dimethylacrylate (TEGDM), poly(ethyleneglycol) dimethylacrylate (PEGDM), polydimethylsiloxane (PDMS), polyethylene glycol succinate, octoxynol, and combinations thereof such as polyethylene glycol succinate and octoxynol, as polymeric matrix containing the compounds for sustained release.

### EXAMPLES

### Diabetes Resulted in Significant Reduction in Retinal Dopamine Level

To assess the effects of diabetes on retinal DA levels, rats were rendered diabetic for either 4 or 12 weeks and their retinal DA levels were compared with age-matched CTRL rats. All rats receiving STZ became hyperglycemic with reduced weight compared with citrate-buffer-treated rats. Overall, DA levels were significantly decreased in DM rats compared with CTRL rats (main treatment effect: F(1,17)=38.233, p<0.001; FIG. 1A). DA levels also increased as the animals aged, regardless of diabetes status (main duration effect: F(1,17)=9.407, p=0.007; FIG. 1A). Moreover, DA reduction worsened from ˜25% at the 4-week time point to 32% at the 12-week time point. Interestingly, only age, not diabetes, altered the levels of DOPAC (main duration effect: F(1,17)=16.796, p<0.001; FIG. 1B). In terms of DA metabolism, the ratio of DA to DOPAC was not significantly altered due to diabetes, but did increase as the animals aged (main duration effect: F(1,17)=15.492, p=0.001; FIG. 1C). Next, whether similar DA reduction occurred in DM mice was assessed. All mice injected with repeated low-dose STZ developed diabetes and maintained it throughout the experiment. More importantly, similar to DM rats, DM mice had significantly lower DA levels, an ˜15% reduction, compared with CTRL mice (Student's t value=2.312, p=0.039; FIG. 2A) at the 5-week time point. No significant differences were found in DOPAC levels or DOPAC/DA ratios between the two groups (FIG. 2B,C).

### Restoring DA Content Delayed Diabetes-Induced Visual Dysfunction

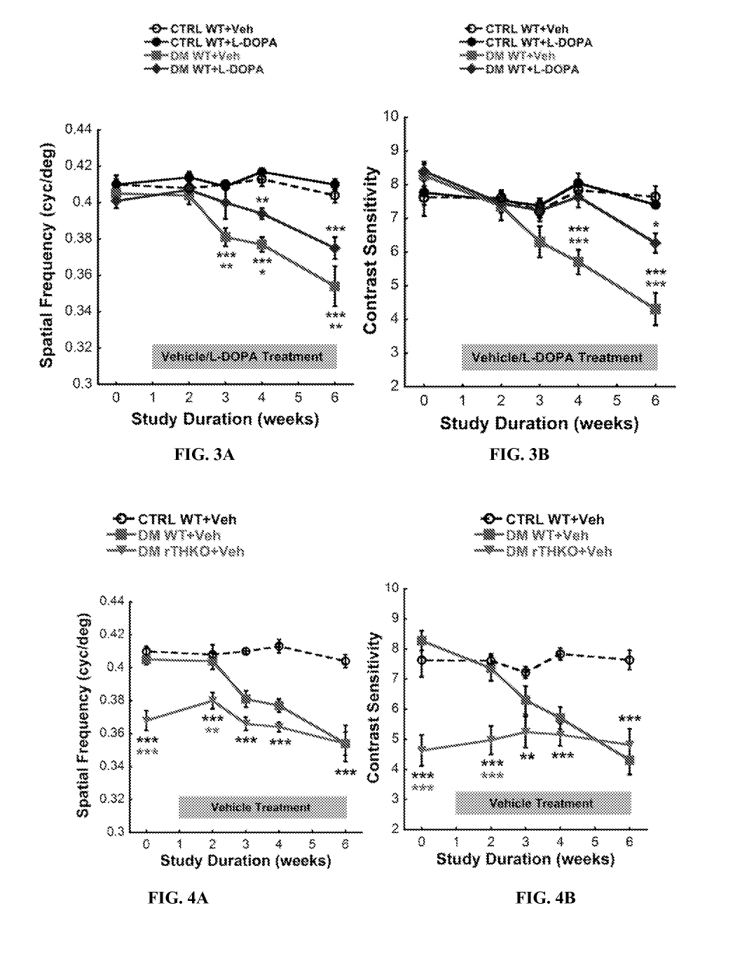

To examine whether DA deficiency causes visual deficits in early DR, the effects of restoring DA levels with L-DOPA on visual function of DM mice was investigated. All STZ-treated DM mice were significantly hyperglycemic compared with CTRL mice. DM WT+Veh mice exhibited significantly reduced spatial frequency thresholds (interaction effect: F(12,82)=4.644, p<0.001; FIG. 3A) and contrast sensitivities (interaction effect: F(12,82)=6.425, p<0.001; FIG. 3B), as early as 3-4 weeks after STZ. Moreover, the severity of visual deficits progressed over time. The deficit in spatial frequency threshold of DM WT+Veh group (compared with CTRL WT+Veh group) worsened from 7.0% at 3 weeks after STZ to 12.3% at 6 weeks after STZ, whereas the reduction in contrast sensitivity deteriorated from 12.8% at 3 weeks after STZ to 43.6% at 6 weeks after STZ. Conversely, the onset and progression of visual dysfunction was significantly delayed by chronic L-DOPA treatment (FIG. 3A-B). The reduction in spatial frequency threshold of DM WT+L-DOPA group progressed from 2.4% at 3 weeks after STZ to 7.2% at 6 weeks after STZ, whereas the deficit in contrast sensitivity was only significant at 6 weeks after STZ, with an 18.0% reduction from CTRL animals. For comparison, L-DOPA treatment in CTRL mice did not cause significant changes in either spatial frequency threshold or contrast sensitivity. Furthermore, behavioral side effects such as dyskinesia with our current dosage of L-DOPA (10 mg/kg daily) in either CTRL or DM mice was not observed. A dosage was selected that was lower than the typical dosage used to induce dyskinesia (25 mg/kg) yet could still increase retinal DA levels.

### Retinal Dysfunction Underlies the Dopamine-Mediated Visual Deficits in Diabetes

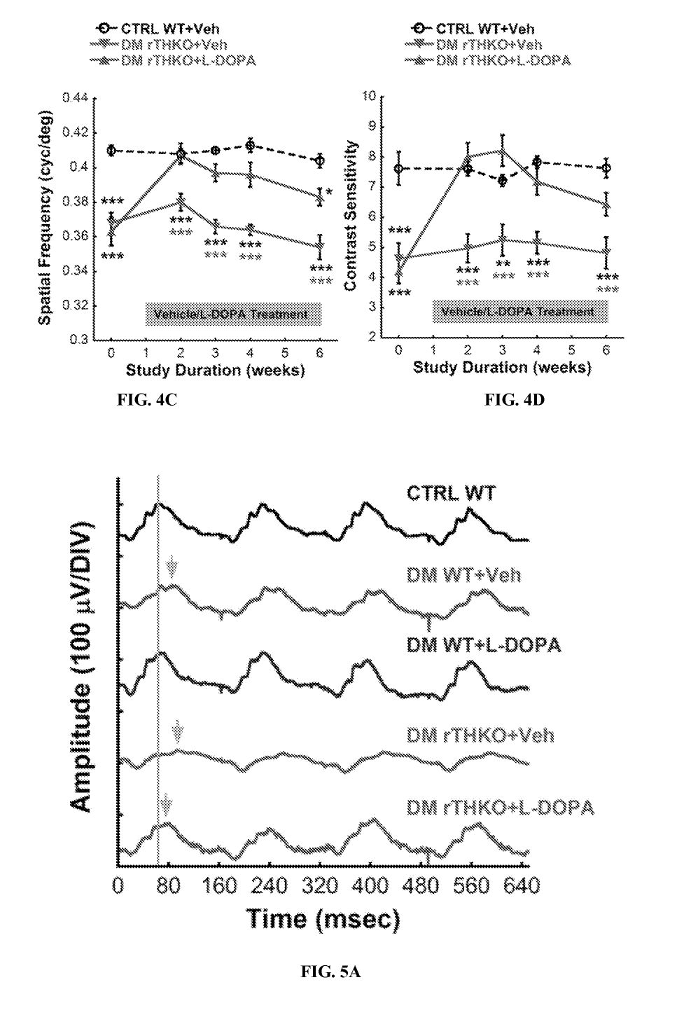

Because systemic injections of L-DOPA affect both brain and retinal dopaminergic systems, the role of retinal DA deficiency underlying visual deficits in early-stage DR was tested by assessing the effects of diabetes in mice with loss of retinal DA (rTHKO mice). It is hypothesized that reduced retinal DA content is a major contributing factor to the early visual deficits, so diabetes in rTHKO mice should not result in a further decline in their visual function. The data indicates that rTHKO mice had reduced spatial frequency thresholds (10.4% lower than CTRL; FIG. 4A,C) and contrast sensitivities (39.3% lower than CTRL; FIG. 4B,D) at baseline (before diabetes induction). More importantly, visual function of the DM rTHKO+Veh group did not further deteriorate after induction of diabetes and maintained the same level of deficit compared with the CTRL group throughout the study period (FIG. 4A,B). Although DM rTHKO+Veh mice began the study with significantly lower spatial frequency thresholds (interaction effect: F(12,95)=8.861, p<0.001; FIG. 4A) and contrast sensitivities (interaction effect: F(12,95)=13.348, p<0.001; FIG. 4B) than those of DM WT+Veh mice, the visual function levels of both groups began to coincide starting at 3 weeks after STZ. To show that DA deficiency could account for the visual dysfunctions observed in DM rTHKO mice, a separate cohort of DM rTHKO mice were treated with L-DOPA. As shown in FIGS. 4, C and D, L-DOPA treatment significantly improved visual functions of DM rTHKO+L-DOPA mice that lasted for the duration of the study.

To validate that daily intraperitoneal injections of L-DOPA were able to increase retinal DA levels, retinal dopamine contents of the following four groups of animals were measured: CTRL WT+Veh, CTRL WT+L-DOPA, DM rTHKO+Veh, and DM rTHKO+L-DOPA. The retinal TH deficiency in the rTHKO mice greatly diminished DA levels compared with both CTRL groups (one-way ANOVA with ranks, H=12.092 with 3 degrees of freedom, p=0.007; Table 3). Conversely, L-DOPA treatment was able to restore the retina DA contents of rTHKO mice to levels comparable to those of CTRL animals. L-DOPA did not significantly increase the DA levels of CTRL mice. Overall, these results provide evidence that retinal DA reduction contributes to the visual defects in early-stage DR and that restoration of retinal DA content with L-DOPA treatment can slow the onset and progression of visual loss.

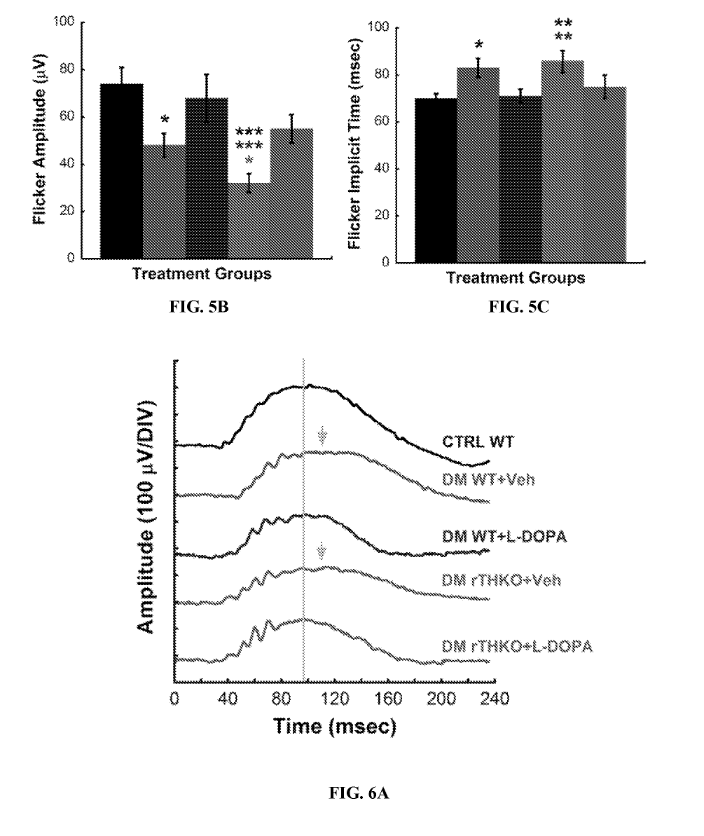

To confirm the hypothesis that retinal dysfunction in early-stage DR contributes to visual deficits and to further support a role for retinal DA deficiency in the visual deficits, whether L-DOPA treatment improved retinal function was investigated as assessed by ERG. Because OKT responses were recorded under photopic conditions, isolated cone pathway function was examined by exposing light-adapted animals to flicker stimuli. The CTRL group shown here and in subsequent RT-PCR analyses includes both CTRL WT+Veh and CTRL WT+L-DOPA animals because no significant differences were detected between them. Flicker responses in DM WT+Veh mice were significantly reduced (interaction effect: F(4,33)=6.032, p<0.01; FIG. 5B) and delayed (interaction effect: F(4,33)=3.621, p<0.05; FIG. 5C) compared with the CTRL animals. More importantly, L-DOPA treatment ameliorated these deficits, restoring the flicker responses of DM WT+L-DOPA mice to levels similar to those of CTRL animals (FIG. 5). In mice with retinal DA deficiency (DM rTHKO+Veh), an even greater reduction (post hoc comparison, p<0.001) and delay (post hoc comparison, p<0.01) in flicker responses were observed compared with CTRL mice (FIG. 5). Once again, administering L-DOPA was able to partially restore cone pathway functions of DM rTHKO mice to CTRL levels (FIG. 5).

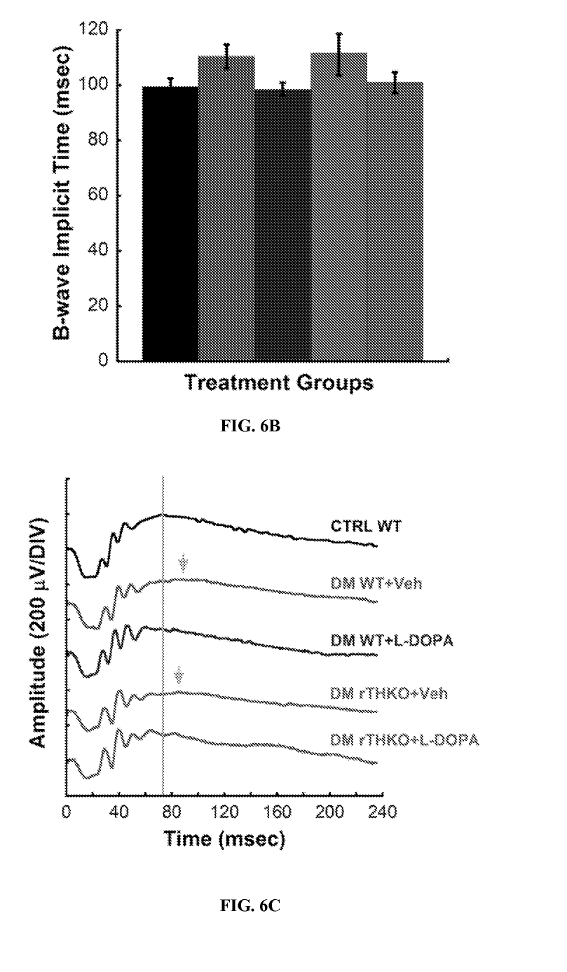

To determine whether L-DOPA treatment was also beneficial for rod pathway function, dark-adapted ERGs were conducted on the same groups of animals. First, no consistent differences were observed in the a-waves, indicating no alterations in the photoreceptoral response at such early stages of diabetes. Next, the postreceptoral function (i.e., b-wave) of these animals was examined. DM WT+Veh animals exhibited significantly delayed b-waves from both CTRL and DM WT+L-DOPA groups under both dim (rods-dominated response: F(2,20)=3.688, p=0.042; FIG. 6A,B) and bright (mixed rods and cones response: F(2,20)=8.311, p=0.002; FIG. 6C,D) flashes. Importantly, L-DOPA treatment reversed the ERG deficits, because no significant difference was detected in the implicit times of the b-waves between CTRL and DM WT+L-DOPA groups. When the experiment was repeated with rTHKO animals, the implicit times of the b-wave responses of DM rTHKO+Veh group under both dim (111.6±8.0 ms) and bright (92.9±8.1 ms) flashes were indistinguishable from those of DM WT+Veh group (dim: 110.4±4.4 ms and bright: 91.1±3.1 ms; FIG. 6). Administration of L-DOPA to rTHKO mice showed a strong, although not statistically significant, trend for improvement in b-wave implicit times under both dim and bright stimuli (FIG. 6). In terms of b-wave amplitude, both groups of DM rTHKO mice (±L-DOPA) showed significantly reduced responses from the CTRL WT animals at both dim (F(4,34)=4.497, p=0.005) and bright (F(4,34)=3.197, p=0.025) flash stimuli (data not shown). The ERG data reinforce the hypotheses that retinal dysfunction underlies early diabetes-associated visual defects and that L-DOPA therapy is able to improve retinal function and thereby slow the progression of visual loss.

### Retinal Transcript Levels of Key Dopamine Proteins Unchanged with Diabetes

Because data indicated that diabetes produced retinal DA deficiency, changes in Th transcript levels may mediate this pathology. Using real-time RT-PCR, it was found that diabetes did not significantly alter Th levels (FIG. 7, Table 4), although L-DOPA treatment resulted in a trend for Th downregulation. The specificity of our Th primers was confirmed, because a >4-fold reduction in Th expression in rTHKO mice (Student's t values: DM rTHKO+Veh=5.964 and DM rTHKO+L-DOPA=5.928, p<0.01; FIG. 7, Table 4) regardless of L-DOPA treatment was found.

Whether diabetes affected transcript levels of DA receptors, specifically D1 receptor (Drd1) and D4 receptor (Drd4), which are selectively involved in spatial frequency threshold and contrast sensitivity was investigated. No significant changes were found in the transcript levels of Drd1 and Drd4 (FIG. 7, Table 4). Interestingly, L-DOPA treatment led to a significant downregulation of Drd4 transcript levels in DM WT mice (Student's t value: DM WT+L-DOPA=2.154, p<0.05; FIG. 7, Table 4), which was consistent with DA-dependent regulation of Drd4 expression levels.

### Selective Improvement in Visual Function with Dopamine Receptor Agonists

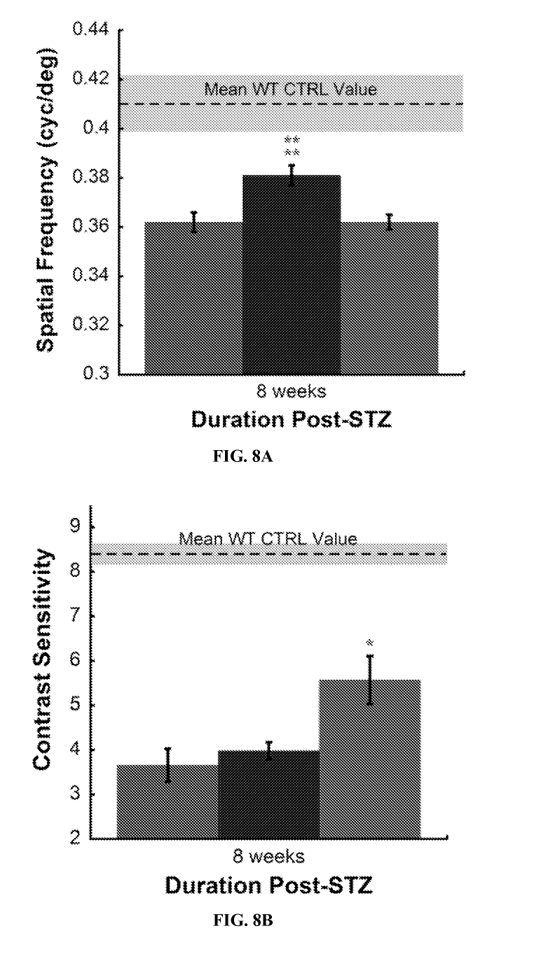

Next, whether activation of DA pathways with selective receptor agonists could reverse visual defects in animals with established diabetes by inducing diabetes was investigated in a group of WT mice with STZ inducement for 8 weeks. After 8 weeks of diabetes, the animals were injected with vehicle, D1 receptor (D1R) agonist (SKF38393), and D4 receptor (D4R) agonist (PD168077). DA agonist treatment was able to restore both spatial frequency threshold (F(2,10)=8.550, p=0.007; FIG. 8A) and contrast sensitivity (F(2,10)=5.321, p=0.027; FIG. 8B), but not to the CTRL WT levels. More interestingly, administration of D1R agonist only improved spatial frequency threshold, whereas administration of D4R agonist only improved contrast sensitivity. These results indicate that treatments targeting the dopaminergic system could be beneficial to patients with established diabetes.

1. A method of treating or preventing visual dysfunction or loss of vision comprising administering an effective amount of dopamine, derivative, ester, prodrug, or salt thereof to a subject wherein the subject is at risk of, exhibiting symptoms of or diagnosed with diabetes or diabetic retinopathy.

2. The method of claim 1, wherein the dopamine derivative is levodopa.

3. The method of claim 1, wherein the dopamine, derivative, ester, prodrug, or salt thereof is administered in combination with an aromatic L-amino acid decarboxylase inhibitor.

4. The method of claim 3, wherein the aromatic L-amino acid decarboxylase inhibitor is selected from carbidopa, benserazide, methyldopa, and α-difluoromethyl-dopa.

5. The method of claim 1, wherein the dopamine, derivative, ester, prodrug, or salt thereof is administered in combination with a catechol-O-methyl transferase (COMT) inhibitor.

6. The method of claim 5, wherein the catechol-O-methyl transferase (COMT) inhibitor is selected from entacapone, tolcapone, and nitecapone.

7. The method of claim 1 comprising administering an effective amount of levodopa in combination with carbidopa and entacapone.

8. The method of claim 1, wherein the dopamine, derivative, ester, prodrug, or salt thereof is administered orally or into the vitreous or sclera of the eye.

9. The method of claim 1, wherein levodopa is administered in combination with fluocinolone acetonide for the treatment of diabetic macular edema.

10. The method of claim 1, wherein the subject is a human.

11. The method of claim 1, wherein the subject is administered dopamine, derivative, ester, prodrug, or salt thereof daily.

12. A method of treating or preventing visual dysfunction or loss of vision comprising administering an effective amount of a dopamine receptor agonist to a subject wherein the subject is at risk of, exhibiting symptoms of, or diagnosed with diabetes or diabetic retinopathy.
